(19) United States
(12) Patent Application Publication
(10) Pub. No.: US 2008/0200917 A1
(43) Pub. Date: Aug. 21, 2008

(54) Targeting device for femur intertrochanteric fracture

(76) Inventor: Shih-Wei Lin, Jhongli City (TW)

Correspondence Address: TROXELL LAW OFFICE PLLC, SUITE 1404, 5205 LEESBURG PIKE, FALLS CHURCH, VA, 22041, US

(21) Appl. No.: 11/706,412

(22) Filed: Feb. 15, 2007

(51) Int. Cl. A61B 17/17

(52) U.S. Cl. 606/96

(57) ABSTRACT

An targeting device for intertrochanteric fracture includes a handle portion and an insertion portion to define an included angle; a protruding portion being formed at a free end of the insertion portion; a through hole being provided on the insertion portion at where closer to its center; the insertion portion being placed into a neck of the femur at where closer to its center; a guide wire being inserted via the through hole for the targeting device to fast and accurately achieve the central fixation at a head of the femur thus to match the femur and the neck of the femur for facilitating subsequent processes of drilling and placement of a dynamic hip screw to firmly secure the femur where the fracture takes place.

### BACKGROUND OF THE INVENTION

(a) Field of the Invention

The present invention is related to a targeting device for femur intertrochanteric fracture, and more particularly, to a mechanism including targeting device and method for femur intertrochanteric fracture that helps a guide wire to accurately arrive at the center of the head of the femur for fast location and match between the femur head and the femur shaft to upgrade precision of dynamic hip screw fixation.

(b) Description of the Prior Art

Whereas the senile people suffering osteoporosis are vulnerable to femur intertrochanteric fracture as illustrated in FIG. 1 of the accompanying drawings, a dynamic hip screw (DHS) is generally used to fix the fracture when the intertrochanteric fracture of a femur 11 takes place. The DHS includes a tube plate 12 and a lag screw 14 that penetrates through the tube plate 12 and is implanted from the outer side of the femur 11 into the femur head 13, and multiple bone screws 15 to fix the tube plate 12 to the outer side of the femur 11.

Before fixing the lag screw 14, a guide wire 16 as illustrated in FIG. 2 is drilled from the lateral side of the femur 11 into the femur head 13. A bone drill 5 is used to place in the guide wire 16 once the femur 11 matches the fracture of the femur head 13 as illustrated in FIG. 3; meanwhile a hole is each drilled into the femur 11 and the femur head 13 with both holes connecting through each other to define a communicating hole 17; the lag screw 14 and the tube plate 12 are inserted; the guide wire 16 is retreated; and the tube plate 12 is fixed to the lateral side of the femur 11 with those screws 15.

In practice, during the insertion of the guide wire 16, the doctor has to insert the guide wire 16 to the center of the femur head 13 from lateral side of the femur 11 while watching the X-ray monitor to make sure the guide wire 16 is driven in the correct direction. It is not easy to align at the central position of the femur head 13 since the guide wire 16 is too thin as indicated by the dotted line in FIG. 2.

If the lag screw fails to reach the central of femur head then the holding power of lag will be decreased. The displacement of the lag screw may fail the operation and may even lead to a more serious complication when the screw pierces through the femur head.

### SUMMARY OF THE INVENTION

The primary purpose of the present invention is to provide a targeting device for the intertrochanteric fracture to help a guide wire fast and accurate reach the center of the head of a femur and rapid match in position between the femur and the head of the femur for improving the locating effect of a DHS.

To achieve the purpose, a targeting device of the present invention is provided with a handle portion and an insertion portion with both portions defining a proper included angle; a free end of the insertion portion relates to a protruding portion; and a through hole is provided on the insertion portion at where closer to its center. The insertion portion enters into where closer to the central position of the neck of the femur where the fracture takes place; a guide wire passes through the through hole and is secured at the center of the head of the femur by means of the targeting device for the femur to match the head of the femur thus to facilitate the subsequent drilling and placement of the DHS for a firm fixation at where the fracture takes place.

### BRIEF DESCRIPTION OF THE DRAWINGS

FIG. 1 is a schematic view showing an operating status of a dynamic hip screw of the prior art.

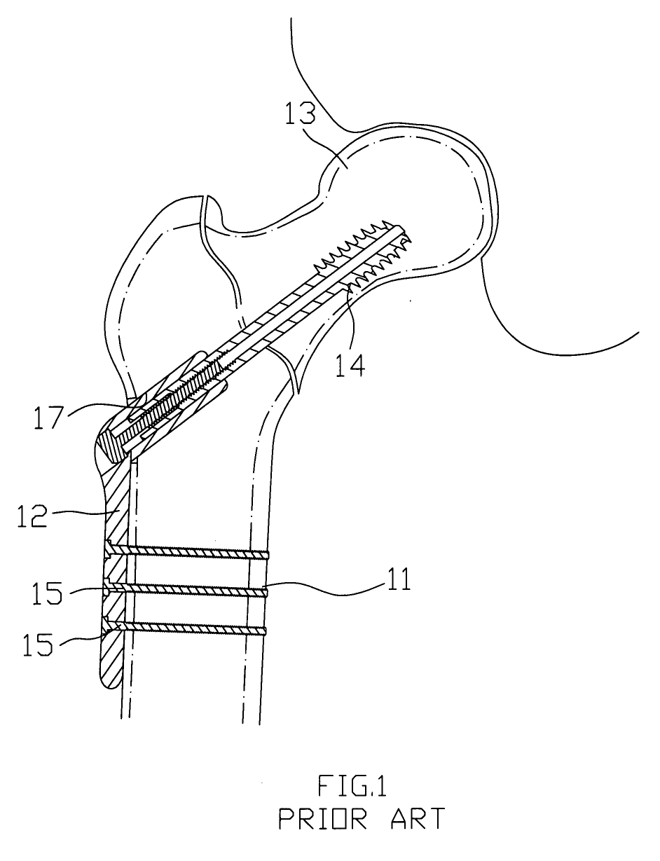

FIG. 2 is a schematic view showing insertion of a guide wire of the prior art into a femur.

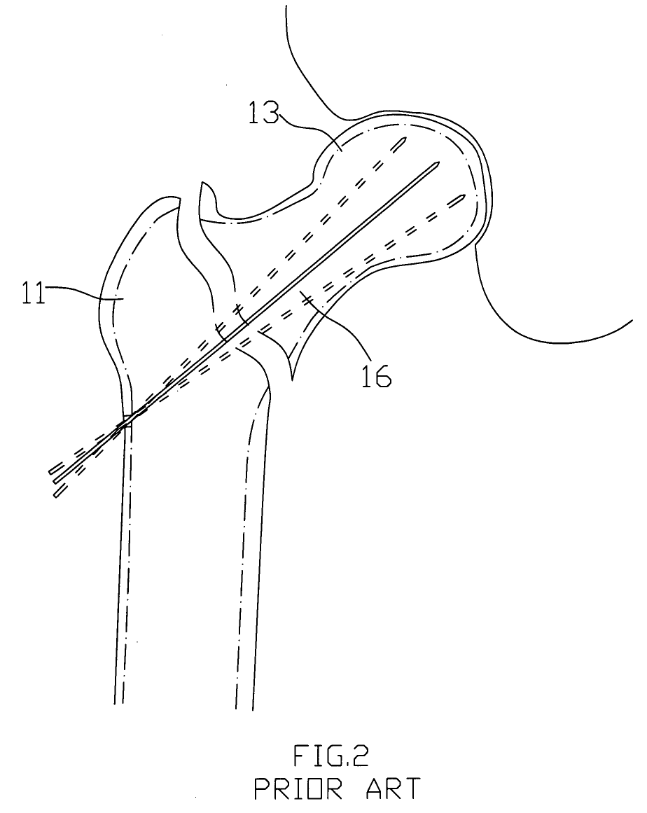

FIG. 3 is a schematic view showing a construction of a common bone drill used in the prior art.

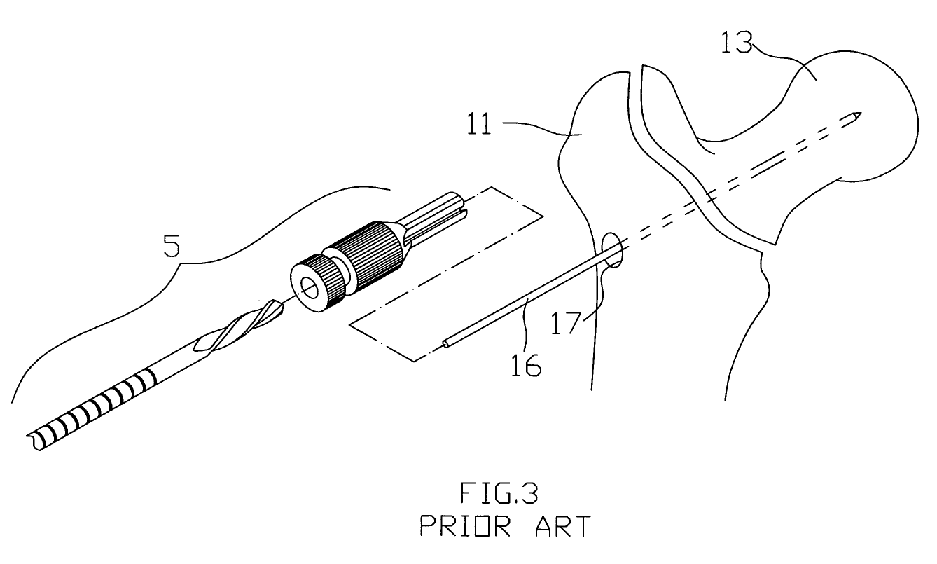

FIG. 4 is a perspective view of a targeting device of the present invention.

FIG. 5 is a schematic view showing a construction of forming a through hole on lateral side of the femur.

FIG. 6 is a schematic view showing a construction of inserting the targeting device of the present invention into a neck of the femur.

FIG. 7 is a schematic view showing a construction of an adjustment of a handle portion to align a protruding portion pointing to the center of femur head in the present invention.

FIG. 8 is a schematic view showing a construction of passing a guide wire through the targeting device of the present invention.

FIG. 9 is a schematic view showing a construction of a common bone reamer used in the present invention.

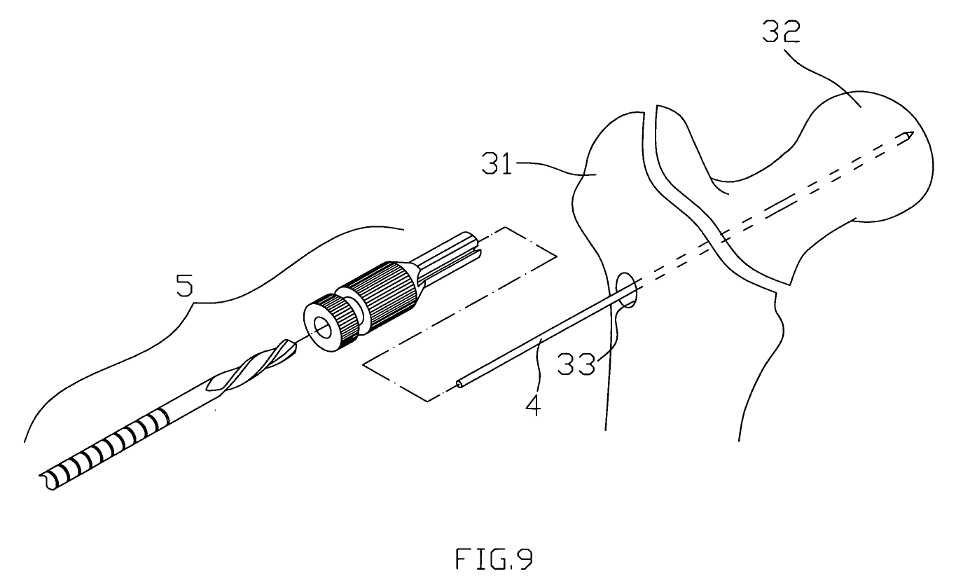

FIG. 10 is a schematic view showing an operating status of a post-operation DHS in the present invention

### DETAILED DESCRIPTION OF THE PREFERRED EMBODIMENTS

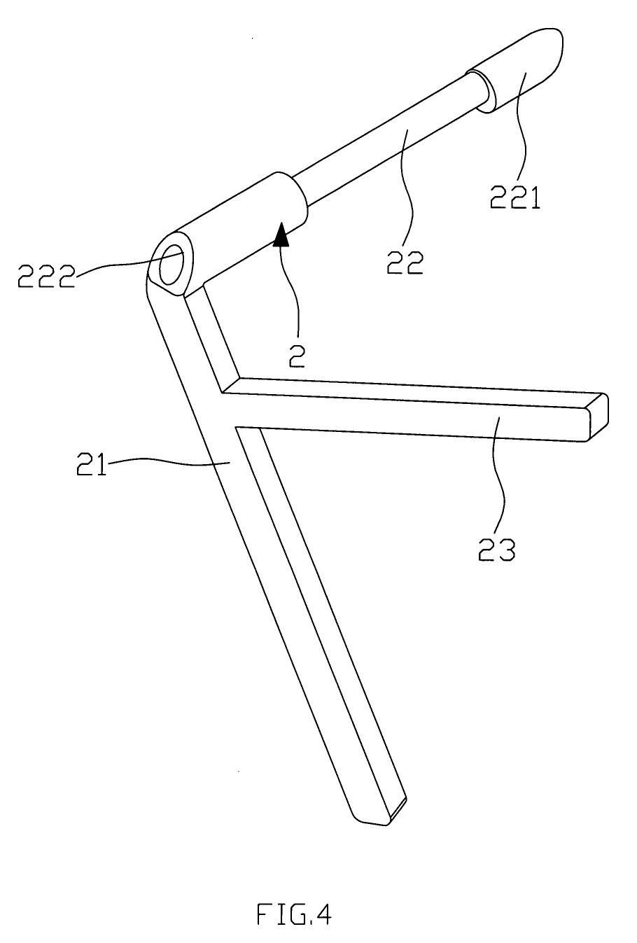

Referring to FIG. 4, an targeting device 2 of the present invention is provided with a handle portion 21 and an insertion portion 22 with both portions to define a proper include angle; a protruding portion 221 is formed on an free end of the insertion portion 22; and a through hole 222 is provided on the insertion portion 22 at where closer to its center.

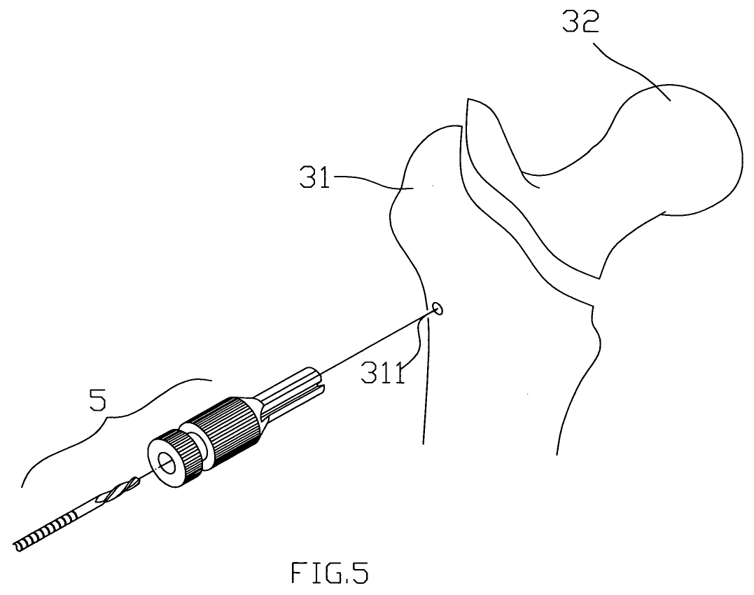

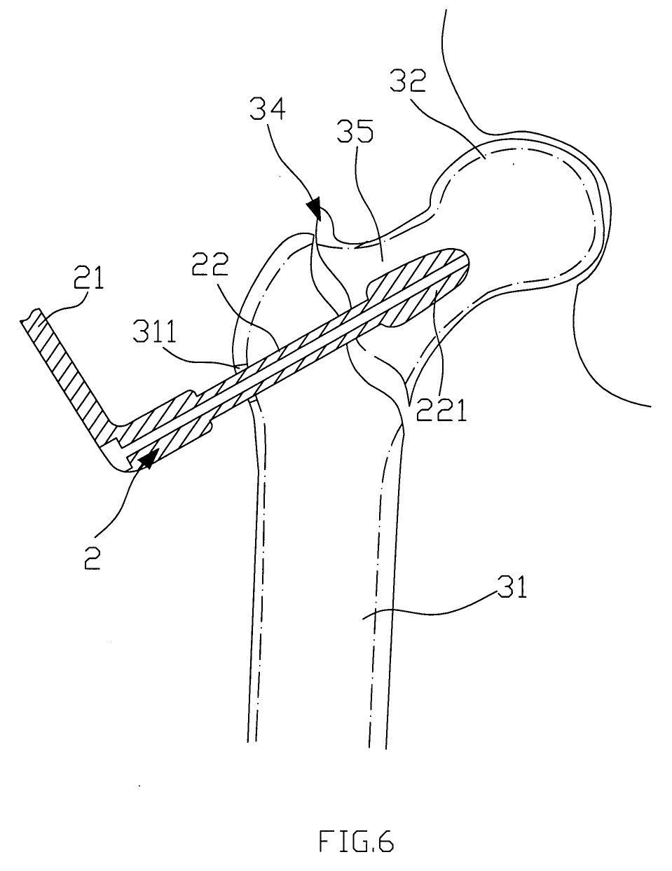

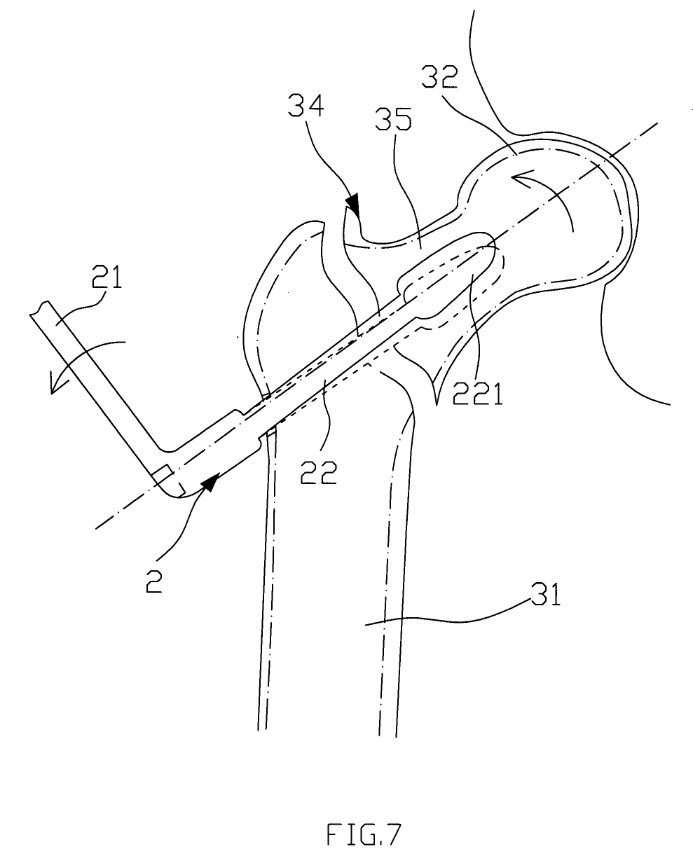

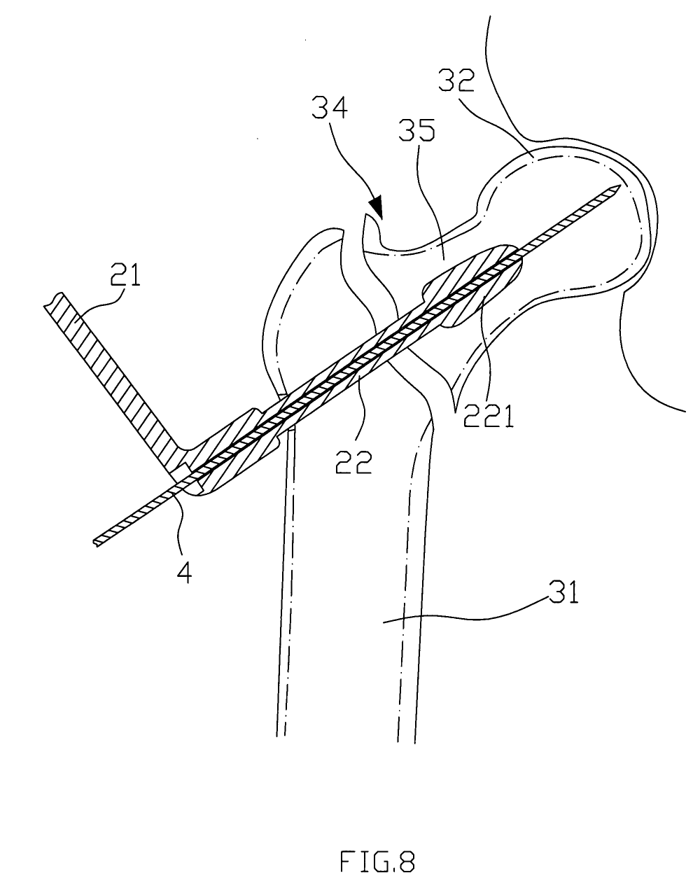

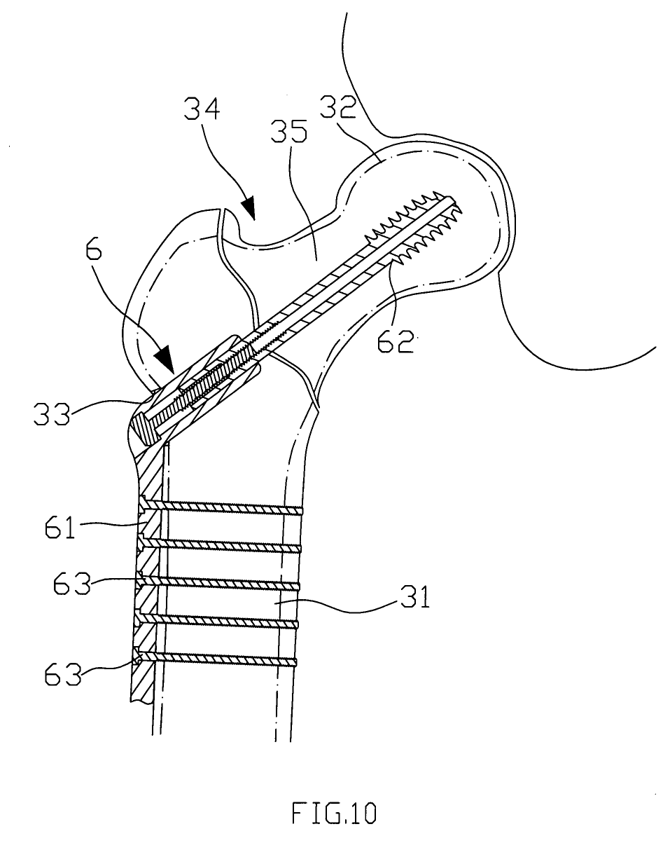

The method to help locating intertrochanteric fracture of the present invention is comprised of the following steps
A. A through hole 311 in a diameter approximately of 13 mm is drilled on lateral side of a femur 31 using a bone reamer 5 also as illustrated in FIG. 5;
B. As illustrated in FIG. 6, the insertion portion 22 of the targeting device 2 is inserted via the through hole 311 into the femur 31 and pushed in the direction of a neck 34 of a head 32 of the femur to enter a neck 35 of the femur;
C. Adjust the position of the targeting device 2 by using the handle portion 21 exposed out of the side of the femur 31 as illustrated in FIG. 7; the handle portion 21 is rotated to adjust the position of the protruding portion 221 such that the protruding portion 221 passes through the neck 35 of the femur and aligns at the central position of the head 32 of the femur;
D. A guide wire 4 is inserted into the through hole 222 to help the head 32 of the femur secure at its central position by means of the targeting device 2 as also illustrated in FIG. 8;
E. A drilling operation is followed by inserting the guide wire 4 into the bone drill 5 as illustrated in FIG. 5 and drilling a hole 33 in the head 32 of the femur that communicates with the femur 31 as illustrated in FIG. 10; and
F. A dynamic hip screw (DHS) is inserted into the communicating hole 33, wherein the DHS 6 is comprised of a tube plate 61, a lag screw 62, and multiple bone screws 63 to secure the bone plate 61 to the shaft of the femur 31; one end of the nail 62 passes from the side of the femur 31 into the head 32 of the femur to firmly secure the femur 31 and the head 32 of the femur where the fracture takes place.

The targeting device for intertrochanteric fracture of the present invention helps fast alignment at the central position of the head of the femur to increase fixation strength, minimize the incidence of complication, shorten the operation time, diminish hemorrhage, and reduce the exposure to radiation for the patient and the medical personnel.

Furthermore, an auxiliary handle portion 23 is further provided to the targeting device 2 as illustrated in FIG. 4. The auxiliary handle portion 23 extends from the handle portion 21 to one side and defines a proper included angle with the handle portion 21 for the user to hold the handle portion 21 with one hand and grip the auxiliary handle portion with another hand to facilitate adjusting the angle of the targeting device 2.

The present invention provides an improved targeting device for femur intertrochanteric fracture, and the application for a utility patent is duly filed accordingly. However, it is to be noted that the preferred embodiments disclosed in the specification and the accompanying drawings are not limiting the present invention; and that any construction, installation, or characteristics that is same or similar to that of the present invention should fall within the scope of the purposes and claims of the present invention.

I claim:

1. A targeting device for intertrochanteric fracture including a handle portion and an insertion portion; a protruding portion being formed on a free end of the insertion portion; and a through hole being disposed on the insertion portion at where closer to the center of the insertion portion.

2. The targeting device for intertrochanteric fracture as claimed in claim 1, wherein the handle portion and the insertion portion define a proper included angle.

3. The targeting device for intertrochanteric fracture as claimed in claim 1, wherein the targeting device is further provided with an auxiliary handle portion.

4. The targeting device for intertrochanteric fracture as claimed in claim 3, wherein the auxiliary handle portion extends from one side of the handle portions and defines a proper included angle with the handle portion.

5. An targeting device for intertrochanteric fracture including the following steps:
A. Place an insertion portion of the targeting device into a femur and push further into a neck of the femur;
B. Adjust the position of the targeting device by using a handle portion exposed out of a side of the femur;
C. Pass a guide wire into a through hole disposed on the targeting device to achieve the purpose for a head of the femur to secure its center in position;
D. Drill a hole that communicates the femur and the head of the femur; and
E. Place a dynamic hip screw in the hole to firmly secure the femur and the head of the femur at where the fracture takes place.

6. The targeting device for intertrochanteric fracture as claimed in claim 5, wherein a bone reamer is used to drill a through hole from lateral side of the femur and place the targeting device into the femur via the through hole before executing Step A.

7. The targeting device for intertrochanteric fracture as claimed in claim 5, wherein the guide wire is inserted into the bone reamer before drilling the head of the femur in executing Step D.

8. The targeting device for intertrochanteric fracture as claimed in claim 5, wherein the dynamic hip screw is comprised of a bone plate, a nail, and multiple bone screws to secure the tube plate to the lateral side of the femur.
